(19) United States
(12) Patent Application Publication
(10) Pub. No.: US 2018/0177579 A1
(43) Pub. Date: Jun. 28, 2018

(54) Body Tissue and Vessel Compression Valve Apparatus

(71) Applicant: Peter Sayet, Fort Lauderdale, FL (US)

(72) Inventor: Peter Sayet, Fort Lauderdale, FL (US)

(21) Appl. No.: 15/848,366

(22) Filed: Dec. 20, 2017

Related U.S. Application Data
(60) Provisional application No. 62/414,450, filed on Oct. 28, 2016.

(51) Int. Cl. A61F 2/00

(52) U.S. Cl. CPC A61F 2/0036; A61F 2250/0002; A61F 2220/0025; A61F 2/004

(57) ABSTRACT

A body tissue and vessel compression valve apparatus devised to position disjointed components remotely locatable into the body of a patient, to minimize obtrusion and discomfort of the patent, and to enable position of a valve head assembly in control of a lumen, or other tissue mass within a patient, while implanting such ancillary components as are required to drive the valve head assembly in separate and distinct locations within the patient whereby access to said components is enabled independently and implantation to a patient's specific physique is accommodated.

Be it known that I, Peter Sayet, a citizen of the United States, have invented new and useful improvements in a body tissue and vessel compression valve apparatus as described in this specification and that this nonprovisional application claims the benefit of provisional application No. 62414450 filed on Oct. 28, 2016.

### BACKGROUND OF THE INVENTION

Various types of body tissue and vessel compression valve apparatuses are known in the prior art. Many of these have been patented by the instant inventor, which patents are hereby incorporated herein by reference: U.S. Pat. Nos. 6,319,191; 6,527,701; 6,689,046; 7,011,621; 7,476,195; 8,360,955; 8,382,652; and 9,144,482. However, what is needed is a body tissue and vessel compression valve apparatus that is implantable in vivo in distal array, each primary component separable and independently installable and accessible, whereby automated closure of a body lumen or compression of a tissue mass, such as the urethra of a patient for example, is effected by remotely situated components interconnected together but separably positionable in a patient's body in vivo, whereby the profile of the device in any particular position is streamlined and less obtrusive for wear, the device thereby adaptable between patients to overcome the varieties imposed by individual cases of incontinence and other conditions wherein selective control over a lumen or tissue mass is desirable, and access to each component independently enables less trauma at each location where surgical implantation of the device is required.

### FIELD OF THE INVENTION

The present invention relates to a body tissue and vessel compression valve apparatus devised for streamlined insertion into the body of a patient whereby distally connected components are fit to a patient's body to minimize trauma (and scarring) to any particular area during installation and removal, to enable independent access when required for maintenance or repair, and to minimize obtrusiveness of any one component for comfort during use. The present body tissue and vessel compression valve apparatus, comprising distally connected independent components, therefore is fittable for use of smaller patients and usable in positions in vivo otherwise inaccessible to embodiments collocated or composed as a single unit. Moreover, each individual component, as set forth herein, may be independently sized for particular use and interchangeably arranged. Thus larger valve head assemblies, for example, are contemplated for use controlling larger lumens or tissue masses in a patient's body without size restrictions otherwise resulting from additional components of the device composed as a single unit.

### SUMMARY OF THE INVENTION

The general purpose of the body tissue and vessel compression valve apparatus, described subsequently in greater detail, is to provide a body tissue and vessel compression valve apparatus which has many novel features that result in a body tissue and vessel compression valve apparatus which is not anticipated, rendered obvious, suggested, or even implied by prior art, either alone or in combination thereof.

The present body tissue and vessel compression valve apparatus has been devised to enable implantation into a patient in distal array, whereby each component required to maintain effective operation of a valve head assembly, selectively effecting opening and closure of a lumen in the patient (such as, for example, the urethra), is separately positioned to minimize the obtrusiveness of the device should it otherwise be assembled as a single unit. Additionally, the various components implantable into a patient are thereby each accessible independently whereby maintenance, replacement, or repair of any one of the several components is effective absent the need to access the other components. Trauma to any one particular area whereat any one of the several components is desirously implanted, then, is minimized.

Further, the present body tissue and vessel compression valve apparatus, by situating the several components distally and separately, enables situation of the valve head assembly at relatively small-scale sites. The remaining components required to drive the device are thence positionable elsewhere, accommodative of the anatomical variances extent between patients and individual cases of incontinence, for example. This enables adapted surgical installation of the present apparatus fitted to each individual patient, including sizing specifically between genders as well as to younger and/or smaller patients or even smaller animals in general. Furthermore, each of the several components is interchangeable for manifold uses. A control of throughput at a plurality of lumens is thus contemplated, such as, for example, mechanical control at the pyloric and cardiac sphincters in patients with chronic gastroesophageal reflux disease, for example, or even, when a valve head assembly is suitably devised, control at the rectal sphincter. Interchange of the particular valve head assemblies, then, devised for operation at a specific bodily lumen for individual patients is contemplated as part of this disclosure.

The present body tissue and vessel compression valve apparatus, therefore, includes a subcutaneous housing distally connected to an abdominal cavity housing which in turn is distally connected to a valve head assembly. The subcutaneous housing is impermeable, constructed of appropriate hypoallergenic material, or coated with a hypoallergenic material or layer, whereby inflammation engendered is reducible. The subcutaneous housing includes a battery interiorly disposed therein. As an alternative to the use of an inductive battery, charging and telemetry communication and command system as primarily anticipated for these devices, the subcutaneous housing may also include interconnection ports, disposed to protrude above the epidermis of a patient, for charging and recharging the battery and for connection with a peripheral device usable to communicate with memory and a processor likewise housed interior to the subcutaneous housing when relevant. Instructions may be uploadable to memory whereby schedules may be uploaded for timed release of the valve head assembly and data may be downloadable from memory as to schedules performed or other data (such as biometrics sensed by the implanted components, such biometrics including, for example, inflammation response at the components, flow data for fluids sensed passing through the controlled lumen, pressure settable and exerted at the valve head assembly, among other such data as may be sensible by the present device when situated in vivo).

The subcutaneous housing may further be situated outside the body of a patient, and may include manual controls to enable controlled release and closure of the valve head assembly. The subcutaneous housing may likewise include a means to wirelessly communicate with a peripheral device, such as a hand held computing apparatus, including, for example, a smart phone, tablet, laptop, or other portable computing device enabled for wireless connection via near field communication protocols, whereby control of the valve head assembly may be effected remotely by manual interaction at the peripheral device. A user may, therefore, make use of a smart phone in said user's possession to effect control of the valve head assembly without directly accessing the subcutaneous housing. In such an embodiment, a transceiver as required to effect near field communication with a peripheral device may be located exteriorly from the body of a patient to minimize exposure of adjacent and overlying tissue to emitted radiation as required to establish near field communication protocols. The device may also use an inductive battery charging system wherein the subcutaneously located battery is charged through epidermal layers using a charging apparatus outside the body.

The subcutaneous housing is disposed distally connected to the abdominal cavity housing. This elongate and streamlined housing is disposed to contain an actuator effecting mechanical control of the valve head assembly. The abdominal cavity housing, therefore, may be sited in the abdomen of a patient, or elsewhere in the body, more proximal the lumen or tissue mass whereat controlled interaction is desired. The abdominal cavity housing may be surgically implanted beneath the endodermis, where appropriate, without concern of potentially toxic components housed in the subcutaneous housing being implanted deeply into the body of the patient. Moreover, size of the abdominal cavity housing is dependent only on the actuator specifications, without need to accommodate the additional components stored in the subcutaneous housing, as in previous embodiments known in the art.

The actuator is contemplated in various forms, including an electric motor operatively communicating with a screw gear and an inside magnet coupler and an outside magnet coupler. The electric motor assembly may also include an iteration wherein the motor is part of a peristaltic pump mechanism capable of controlling the forward or reverse flow of fluid in a closed fluid system assembly that would include a reservoir at the distal end of the fluid system and a cuff at the proximal end of the fluid system that will act as the compression force on the body lumen/vessel or tissue mass. Magnetic induction is likewise contemplated, whereby a particular current is effective to create a proportionate magnetic field to attract the coupler and mechanically transfer motion at a regulated pressure to the valve head assembly.

The valve head assembly maintains a collapsible aperture distally disposed relative the abdominal cavity housing. The valve head assembly is therefore limited in scale only by the diameter of the aperture desired. The valve head assembly is thereby enabled position surgically implanted surrounding a particular lumen in a patient at nearly all scales.

Action of the actuator in the abdominal cavity housing effects movement of the valve head assembly between an open position, whereby the lumen in question is unobstructed, and a closed position, whereby the lumen in question is obstructed and free flow of fluids therethrough is prevented. Control of the valve head assembly is thus enabled distally relative the location of the lumen in question and implantation of the abdominal cavity housing is effective at smaller scales than if the battery, memory, interconnection ports, and other such components be collocated therewith. Situation of the subcutaneous housing distally relative the abdominal cavity housing thus enables preferential siting of the subcutaneous housing particular to each patient, enabling better access and control of each of said components, adapted to each individual case, as desired.

Thus has been broadly outlined the more important features of the present body tissue and vessel compression valve apparatus so that the detailed description thereof that follows may be better understood and in order that the present contribution to the art may be better appreciated.

For better understanding of the body tissue and vessel compression valve apparatus, its operating advantages and specific objects attained by its uses, refer to the accompanying drawings and description.

### BRIEF DESCRIPTION OF THE DRAWINGS

### FIGURES

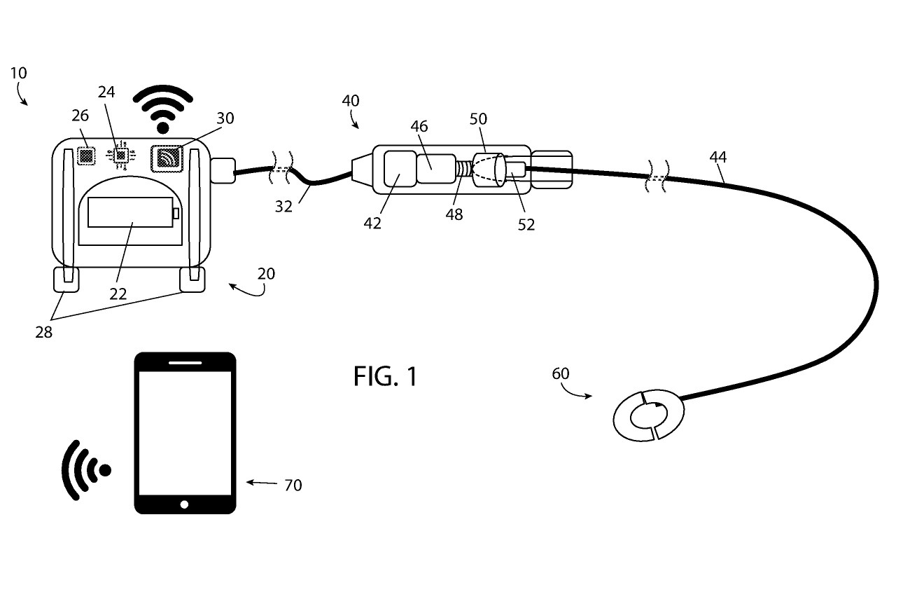

FIG. 1 is a diagrammatic view of an example embodiment.

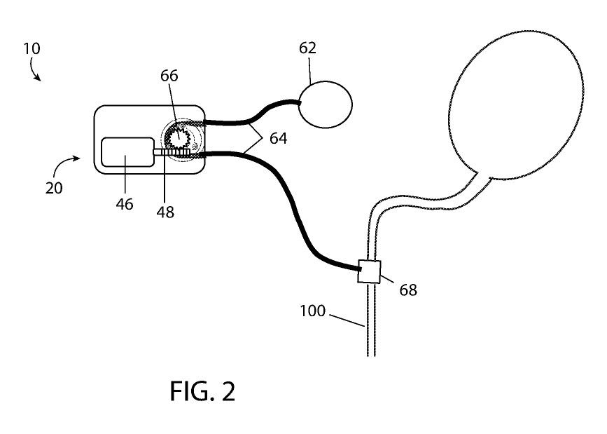

FIG. 2 is a diagrammatic view of an example embodiment.

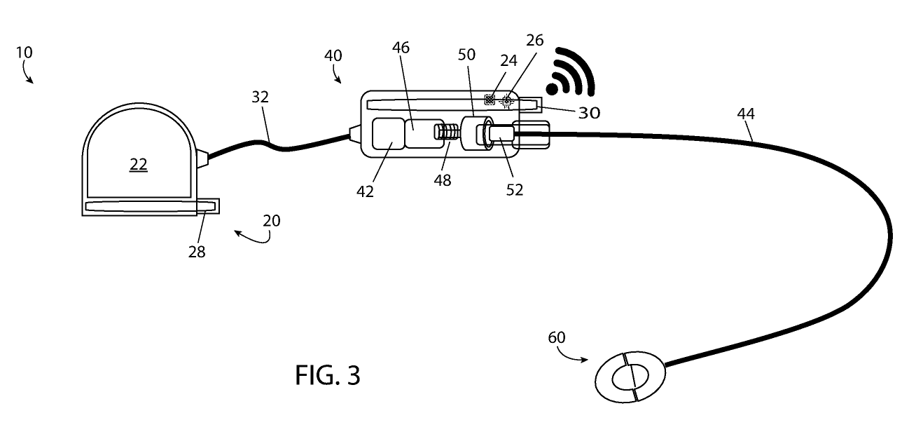

FIG. 3 is a diagrammatic view of an example embodiment.

### DETAILED DESCRIPTION OF THE DRAWINGS

Referring to FIGS. 1 and 3, an example embodiment of the body tissue and vessel compression valve apparatus is illustrated.

In the present invention 10 a battery 22 is contained interior to an impermeable subcutaneous housing 20, said subcutaneous housing 20 distally disposed relative an abdominal cavity housing 40, which abdominal cavity housing 40 is likewise distally disposed relative a valve head assembly 60 configured for situation girding the urethra (or other lumen) of a patient.

The subcutaneous housing 20 is implantable in the body of a patient in a situation removed from the abdominal cavity housing 40 and the valve head assembly 60. The size of the subcutaneous housing 20 is thereby restricted only to dimensions required to house the battery 22 and memory 24 and a processor 26, when present. The separate configuration of the abdominal cavity housing 40 and the valve head assembly 60 enables the subcutaneous housing 20 to be considerably less obtrusive when implanted at a chosen location subcutaneously within a patient. Moreover, maintenance of each of the components is thereby effective independent of the need to access the others, and trauma (and scarring) effected during surgery is considerably diminished at each location whereat a component of the present 10 invention is implanted.

Access to the subcutaneous housing 20 is thereby effective independent of the need to remove the abdominal cavity housing 40 or the valve head assembly 60, each of which may likewise be accessed independently.

The subcutaneous housing 20 is more readily positioned implanted upon a patient in situation for interaction with additional elements, such as a power source to recharge the battery 22. The subcutaneous housing 20 may therefore include interconnect ports 28 devised to protrude exteriorly through the epidermis of a patient for interconnection and communication with peripheral devices 70. The battery 22 may be recharged by connecting at least one of the interconnection ports 28 with an exteriorly available power source and communication between the present device 10 and an interconnected peripheral device 70 may be likewise effected. Moreover, for embodiments of the present invention 10 where wireless communication is contemplated with a proximal peripheral device 70, situation of any transceiver 30 required to establish near field communication with said peripheral device 70 may be oriented exteriorly relative the tissue of a patient whereby any potential damage to overlying and adjacent tissue by emission of radiation (such as, for example, microwaves or high frequency radio waves) is alleviated.

The subcutaneous housing 20 is connected to the abdominal cavity housing 40 by means of a wire connection assembly 32, said wire connection assembly 32 having a length appropriate to enable implantation of the abdominal cavity housing 40 more proximal the lumen whereat articulated closure is to be effected by operation of the present device 10, such as the urethra of the patient. The abdominal cavity housing 40 is likewise impermeable and hypoallergenic and includes an actuator 42 disposed in operational communication with the valve head assembly 60 by means of a distal wire member 44, as will be described subsequently.

The valve head assembly 60, which is distally located endwise on the distal wire member 44 and surgically disposed surrounding a lumen of the patient, for example the urethra, is activated by the actuator 42 and thereby moveable between an open position and a closed position. The valve head assembly 60 is configured to operate at enough pressure to effect closure of the lumen sufficiently to prevent flow of fluids therethrough, but at a pressure maintained beneath a threshold whereby injury is preventable.

The actuator 42 may include a motor 46 operating a screw gear 48 in operational communication with an inside magnet coupler 50 and an outside magnet coupler 52 disposed in operational communication with the valve head assembly 60. Alternatively the actuator 42 may operate by means of magnetic induction communicable along the distal wire assembly 44 whereby the valve head assembly 60 is moveable between the open and closed positions.

It is further contemplated as part of this invention 10 that control of the instant body tissue and vessel compression valve apparatus 10 may be enabled by manual action effective at a peripheral device 70. A user may, therefore, operate a peripheral device 70, such as a handheld computing device or other wireless communications device, to operate the valve head assembly 60 when required. Alternatively manual action to an exposed portion of the device 10, or at a dermally proximal portion of the device 10, may be effected to operate the valve head assembly 60 and thereby enable drainage of fluid otherwise prevented from free-flow through the lumen targeted by the valve head assembly 60.

A patient suffering from incontinence, for example, is thereby enabled selective mechanical operation of the device 10 to open and close the valve head assembly 60 and selectively enable flow of fluids when desired.

Referring to FIG. 2, an embodiment is contemplated that hydraulically effects closure of the valve head assembly 60, rendered as a cuff 68, in this embodiment. Fluid in a fluid reservoir 62 is forcible in each of a first and second direction along a fluid system tubing 64 disposed in fluid communication between the reservoir 62 and the cuff 68. A peristaltic pump 66 effects forcing of the fluid in each of the first and second direction. Flow of the fluid effected in the first direction pressurizes the cuff 62 and effects closure around the targeted lumen or tissue mass 100 (illustrated as an urethra in FIG. 2). Forcing flow of fluid in the second direction effects opening of the cuff 68 and releases the targeted lumen or tissue mass.

Referring to FIG. 3, an embodiment is provided wherein the processor 26, memory 24 and transceiver 30 are positioned interior to the abdominal cavity housing 40 in stead of in the subcutaneous housing 20. In some embodiments, therefore, siting individual components interior to the abdominal cavity housing 40 may be preferable relative the subcutaneous housing 20, depending on the particular patient and the desired implanting sites of each of the components desirously implanted to control opening and closing a bodily lumen or other tissue mass. Thus the fungibility of each of the components and parts of the present apparatus is readily afforded rendering the device 10 adaptable to a variety of uses across a diverse patient population.

What is claimed is:

1. A body tissue and vessel compression valve apparatus comprising:
a fungible impermeable subcutaneous housing having an internally disposed battery;
a fungible abdominal cavity housing having an internally disposed actuator therein, said abdominal cavity housing disposed in operational communication with the subcutaneous housing, said abdominal cavity housing disposed for implantation inside a patient at a site remotely located relative the subcutaneous housing;
a fungible wire connection assembly disposed connecting the subcutaneous housing to the abdominal cavity housing;
a fungible valve head assembly disposed in operational communication with the abdominal cavity housing, said valve head assembly moveable between an open position and a closed condition;
a fungible distal wire member disposed interconnecting the abdominal cavity housing and the valve head assembly;
a processor;
a memory disposed in operational communication with the processor; and
a transceiver disposed in operational communication with the processor and memory;
wherein the valve head assembly is implantable interior to a patient to girdle a lumen or tissue mass, the abdominal cavity housing is implantable in a patient remotely located relative the valve head member, and the subcutaneous housing is implantable in a patient remotely located relative the abdominal housing, whereby selective control opening and closing the lumen or tissue mass is enabled via disjointed area of operative parts.

2. The body tissue and vessel compression valve apparatus of claim 1 wherein the to abdominal cavity housing further comprises a motor, a screw gear, an inside magnet coupler, and an outside magnet coupler.

3. The body tissue and vessel compression valve apparatus of claim 2 wherein the subcutaneous housing further comprises a pair of interconnection ports disposed for interaction with an power source.

4. The body tissue and vessel compression valve apparatus of claim 3 wherein the interconnection ports are disposed to protrude above the epidermis of a patient for interconnection with existing peripheral devices whereby data transfer between the memory, processor, and said existing peripheral devices is enabled.

5. The body tissue and vessel compression valve apparatus of claim 3 wherein the transceiver is disposed in wireless communication with an existing peripheral device whereby the memory and processor is accessible and controllable by action input at the peripheral device and wireless data exchange between said existing peripheral device, said memory, and processor is enabled.

6. The body tissue and vessel compression valve apparatus of claim 3 wherein the memory, processor, and transceiver are disposed in the subcutaneous housing.

7. The body tissue and vessel compression valve apparatus of claim 3 wherein the memory, processor, and transceiver are disposed in the abdominal cavity housing.

8. A body tissue and vessel compression valve apparatus comprising:
an impermeable subcutaneous housing implantable into a patient;
a peristaltic pump disposed inside the housing;
a motor disposed inside the housing, said motor disposed in operational communication with the peristaltic pump;
a fluid reservoir disposed in operational communication with the peristaltic pump, said fluid reservoir remotely disposed relative the subcutaneous housing;
a cuff remotely disposed relative the subcutaneous housing, said cuff disposed for implant within a patient in a position around a lumen or tissue mass of said patient, said cuff expansible when pressurized by fluid forced thereinto, said cuff thereby moveable between an open position and a closed position by action of fluid forced into and respectively out of said cuff; and
a fluid tubing system disposed in open communication between the fluid reservoir, the peristaltic pump, and the cuff;
wherein fluid disposed interior to the fluid reservoir is forcible in each of a first direction and a second direction by action of the peristaltic pump
whereby the cuff is forced between the open and closed position by action of the peristaltic pump.
